(19) United States
(12) Patent Application Publication
(10) Pub. No.: US 2006/0206055 A1
(43) Pub. Date: Sep. 14, 2006

(54) Short-tapered epidural injection needle (ice needle)

(76) Inventor: Dennis Alan Ice, Amarillo, TX (US)

Correspondence Address: DENNIS A. ICE, M.D., 3215 Bowie Street, Amarillo, TX, 79109, US

(21) Appl. No.: 11/369,298

(22) Filed: Mar. 7, 2006

Related U.S. Application Data
(60) Provisional application No. 60/659,587, filed on Mar. 8, 2005.

(51) Int. Cl. A61M 5/178

(52) U.S. Cl. 604/164.01; 604/272

(57) ABSTRACT

An epidural injection needle with a larger-gauge proximal shaft (16, 30) that tapers in close proximity (20, 34) to a smaller-gauge distal tip (22, 38). A conventional stylet (10, 18, 32) of suitable material is employed to stiffen the needle and to prevent material from entering the needle during insertion. Wings (14) can be fitted to facilitate manual guidance of the needle. The short-tapered design provides an epidural injection needle having a shaft large and stiff enough so that it can be reliably guided to the epidural space but also having a small tip which minimizes trauma to blood vessels, nerves, and other tissues that may be inadvertently punctured during the procedure. This enables epidural injections to be safer for patients than those utilizing commercially available needles or prior-art designs.

### CROSS-REFERENCE TO RELATED APPLICATIONS

This application claims the benefit of provisional patent application Ser. No. 60/659,587, filed Mar, 8, 2005 by the present inventor.

### FEDERALLY FUNDED RESEARCH

Not Applicable

### SEQUENCE LISTING OR PROGRAM

Not Applicable

### BACKGROUND OF INVENTION

1. Field of Invention

This invention relates to medical instruments, specifically to needles for performing epidural injections or anesthesia.

2. Prior Art

The epidural space is located between the outer ligamentous structures of the spinal canal and the inner dural sheath (dura mater), which covers the spinal cord and the lumbar nerve bundle (cauda equina). This space is easily reached by an epidural needle for the placement of medications for pain control and reduction of inflammation.

Standard commercially available epidural needles are of one gauge size (diameter) from the proximal (near) end to the distal (far) end. A large size needle allows for good directional controllability, because it is more rigid. It goes where it is pointed. But a large needle can create larger defects in the tissues it penetrates, and can cause injury to nerves, blood vessels, and the dural sheath. On the other hand, a smaller-gauge needle will cause less tissue trauma, but can be very difficult to steer to the target. These smaller-gauge needles may wander unpredictably, endangering tissue structures remote from the intended needle track. Thus both large- and small-gauge needles can cause tissue injury for different reasons.

This invention provides a safer alternative to currently available epidural needles. A larger-gauge proximal tubular shaft allows the needle to be steered accurately. Since the needle tapers a very short distance from the distal end, it minimizes the possibility of serious tissue trauma. In effect, this needle combines the best features of both large-and small-gauge needles while reducing the possibility of untoward tissue injury.

A stepped needle, U.S. Pat. No. 4,781,691 to Gross (1988) was proposed to provide safer injections through the dural sheath into the subarachnoid space itself. Another similar needle, U.S. Pat. No. 5,002,535 to Gross (1991) is a similar device with one step in the needle's length. Placement of medication in the subdural/subarachnoid space is known as spinal anesthesia, as opposed to the solely epidural placement of anesthetic or anti-inflammatory medications. This needle tapers gradually starting at a distance from the distal end and has a sharp, beveled, non-rounded tip.

The Ice Needle provides a much shorter taper at the distal end of the needle. This is an advantage because it results in maximal stiffness for most of the needle's length. The Gross Needle tapers farther from the end, allowing the longer narrow-gauge portion to divert should it hit bone, scar tissue, or other firm tissue structures.

Another advantage of the Ice Needle over the Gross Needle is that the tip of the Ice Needle, in best mode, is rounded or semi-rounded. This feature further reduces the chance of tissue trauma or puncture.

A further advantage concerns the puncture of the dural sheath. When this sheath is punctured, spinal fluid may leak. These leaks frequently heal without problems. Occasionally, they may leak persistently, causing a post-dural puncture headache (PDPH). A larger, sharp needle tip may create a bigger defect in the dural sheath, making these headaches more likely. The smaller tip on the Ice Needle, if inadvertently placed through the dura, will create a much smaller puncture defect, making the possibility of PDPH much less likely because less fluid will leak.

Another device, U.S. Pat. No. 4,405,314 to Cope (1983) specifies a needle whose lumen (hollow part of the tubular shaft) tapers toward the distal end. It also has a side port for the placement of a guidewire. The Ice Needle, as noted, has a large gauge tubular shaft extending almost to the distal end of the device. A needle with a gradual proximal-to-distal taper would tend to flex during insertion and veer off course if it contacts scar tissue or other firm structures. The large, constant-diameter proximal shaft of the Ice Needle provides better directional control and is safer, as it is less likely to divert into blood vessels, nerves and other vulnerable structures. In addition, the Cope Needle is more complex. It has a provision for the insertion of a guidewire during needle placement. This increases complexity, cost of manufacture, and difficulty of use by the operator.

Another device, U.S. Pat. No. 5,669,882 to Pyles (1977) specifies an epidural needle with a stylet that has a curved distal section. The Ice Needle has a straight course from proximal to distal. This makes it easier to guide to the epidural space. The short taper of the Ice Needle is not a feature of the Pyles Needle.

Another advantage over previously-described tapered needles is that the Ice Needle is primarily designed for injections into the epidural space. Other needles have been designed for the passage of epidural catheters. For these applications, beveled tips are frequently used. For purposes of injection, a blunter, less traumatic needle tip is a better, safer choice.

In addition, the Ice Needle is less complex in its design than the other needles mentioned and so should be less expensive to machine and manufacture.

### OBJECTS AND ADVANTAGES

Accordingly, several objects and advantages of my invention are:
(a) to provide an epidural injection needle with sufficient gauge size to be reliably guided into the epidural space;
(b) to provide an epidural injection needle with a smaller-gauge distal end to minimize tissue trauma;
(c) to provide an epidural injection needle with a variety of needle tip types, including, but not limited to; the Tuohy, Crawford, Coude, RxCoude, and Lutz types; and including those having blunt and semi-blunt tips.
(d) to provide an epidural injection needle with a variety of gauge sizes and variations between the larger proximal shaft and the smaller distal tip;
(e) to provide an epidural injection needle with a very short taper from the proximal tubular shaft to the distal end, enhancing the ability to reliably guide the needle to its target;
(f) to provide an epidural injection needle primarily for medication placement into the epidural space;
(g) to provide an epidural injection needle for epidural catheter placement if manufactured with an appropriate distal tip;
(h) to provide an epidural injection needle for cervical, thoracic, lumbar, and caudal use;
(i) To provide an epidural injection needle that is safer for patients than are currently available epidural needles.

Further objectives/advantages of my invention will become apparent from consideration of the drawings and ensuing description.

### SUMMARY

In accordance with the present invention an epidural injection needle comprising a larger-gauge proximal tubular shaft with a short taper at the distal end resulting in a smaller-gauge aperture at the terminal portion of the distal tip. This needle design is proposed in order to enhance patient safety over commercially available and prior art epidural injection needles.

### DRAWINGS—FIGURES

FIG. 1 is a drawing of the entire needle, top/bottom view, approximately 2-2.5× actual size.

FIG. 2 is a drawing of the entire needle, side view, approximately 2-2.5× actual size.

FIG. 3 is a larger scale (enlarged view) drawing of the parts of the needle pertinent to this patent, top/bottom view, approximately 10-15× actual size.

FIG. 4 is a larger scale (enlarged view) drawing of the parts of the needle pertinent to this patent, side view, approximately 10-15× actual size.

### DRAWINGS—REFERENCE NUMERALS

10 proximal end of stylet
12 proximal hollow hub of needle
14 wings for manual guidance
16 proximal shaft of needle
18 shaft of stylet
20 short taper of needle at distal end
22 tip of needle
24 distal opening of needle with stylet shaft intact
30 proximal shaft of needle (enlarged view)
32 shaft of stylet (enlarged view)
34 short taper of needle at distal end (enlarged view)
36 distal opening of needle with stylet intact (enlarged view)
38 tip of needle (enlarged view)

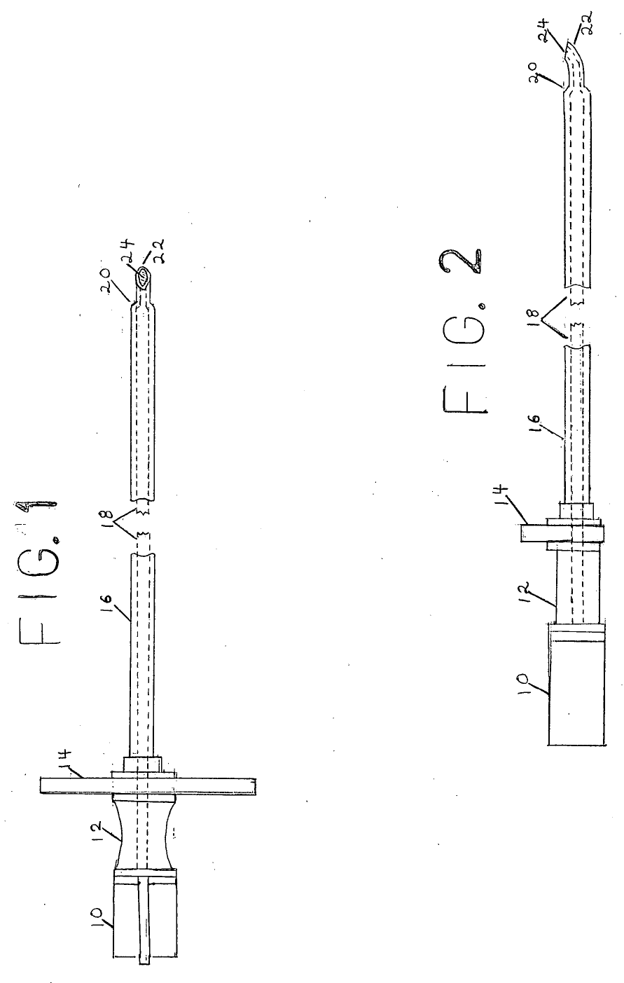

### DETAILED DESCRIPTION—FIGS. 1, 2,3 AND 4-PREFERRED EMBODIMENT

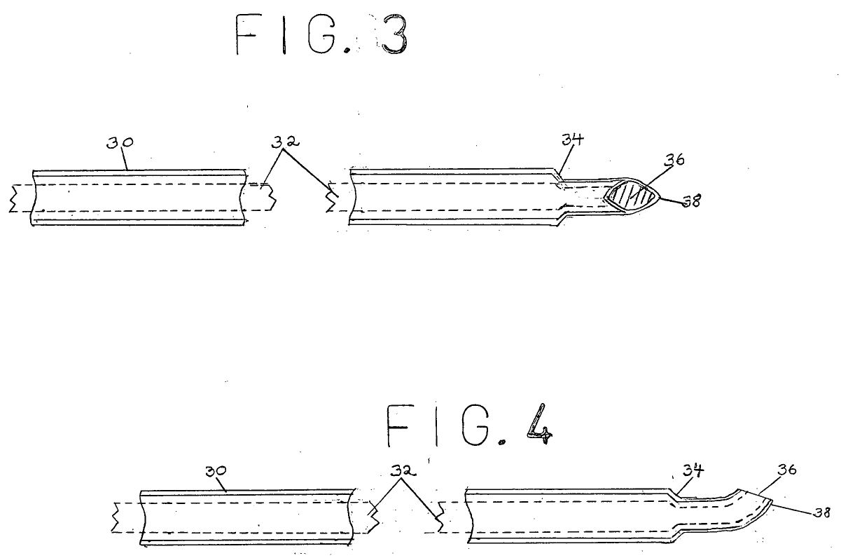

Referring to FIGS. 1 through 4; the device has a proximal hollow hub 12 through which a stylet 10, 18, 32 is passed to the end 24, 36 of the device. A set of wings 14 is attached to the needle hub 12. FIGS. 1 and 2 are complete illustrations of the needle/stylet complex and are 2-2.5× actual size. FIGS. 3 and 4 are enlarged view partial illustrations of the device from the mid shaft to the end with the stylet in place that more clearly show the distal short-taper 34 and the tip 38, and are approximately 10-15× actual size. This view also illustrates -the relationship of the shaft, taper, and the distal end of the needle.

Operation-FIGS. 1, 2, 3, 4

The stylet prevents solid material from entering the needle during insertion and clogging the needle bore. The stylet is flush-fit to the contour of the inner shaft of the needle. It is manipulated by a plastic housing 10 on its proximal end. For clarity, the figures show a gap between the stylet 18 and needle 16. In reality, this space is almost nonexistent. The needle is inserted into the skin with the stylet in place. A pair of wings 14 allows better needle control during insertion. An experienced physician can discern by feel when the needle passes into the ligaments, as resistance to insertional force increases. At this point, the stylet 10, 18, 32 is withdrawn from the needle. The needle remains in the ligament. A glass or low-resistance syringe is then placed on the proximal end of the needle. This syringe has 2-5 cc of air and sterile saline. When the needle tip 22, 38 is in a ligament or other tissue, pressure on the plunger of the syringe compresses the air. A feeling of bounce is perceived by the physician as the air, having no place to go, will not pass out of the needle due to tissue resistance. When the needle tip passes through the deepest ligament, the ligamentum flavum, resistance disappears, and the plunger is depressed as the air enters the open epidural space. At this point, the short-tapered part of the needle 20, 34 and tip 22, 38 are in the epidural space. The treating medication can now be placed into the epidural space. The needle shaft 16, 30 is then withdrawn completely at the end of the procedure.

In the preferred embodiment, the shaft is in the 16-20 gauge range in order to render sufficient stiffness, thus allowing the needle to go where it is pointed instead of veering off track. At the end of the proximal shaft 16, 30, there is a short-tapered portion 20, 34 which decreases the needle diameter at the distal tip 22, 38 to a range of 18-26 gauge, depending on proximal shaft diameter. Smaller tip sizes could be used, but these might be prone to bending or breaking if bone is forcibly contacted. The length of the needle from taper to end in best mode is 0.5-1.0 cm. This dimension is exaggerated in the drawings for the sake of clarity. The tip 22, 38 is shown in the figs with the stylet 24, 36 in place. Tip types may vary, but the best mode employs a rounded sharp or blunt tip to reduce the possibility of tissue trauma. The length of the needle/stylet unit is not specified but may vary as needed due to patient size or other contingencies.

### FIGS. 1-4—Alternative Embodiments

Alternatively, a variety of needle tip types can be used. These may include the Tuohy, Crawford, Coude, RxCoude, Lutz, and others. As noted, a variety of gauges may be used. For example, a 17 or 18 gauge (large diameter) proximal tubular shaft can be mated via short taper to a 22 gauge tip. Best mode calls for no greater than a 2-4 gauge decrease from proximal to distal. This minimizes flow problems that could occur in attempting to push fluid from a large bore shaft through a very narrow taper into a small tip. This will also reduce the resistance that must be overcome in pushing fluid through the needle. A 2-4 gauge reduction in needle diameter will also minimize any difficulties in discerning loss of resistance during the procedure. A greater diameter reduction and/or an excessively small tip may cause measurement of loss of resistance to be unreliable. Also, a 2-4 gauge reduction between shaft and tip would tend to limit the exit velocity of the fluid bolus into the epidural space, making the procedure more comfortable for the patient.

Although this needle has been described as a means to facilitate safe epidural injection, a suitable tip could allow epidural catheter placement. The tapered portion of the needle may be more rounded than shown in the illustrations in order to allow smooth tissue passage. The sharp taper angles illustrated are for the sake of clarity, as is the exaggerated distance from the taper to end of the needle. The needle may be constructed of a variety of conventionally-utilized materials. The hub may be of plastic, metal, or other suitable material. The needle itself may-be constructed of stainless steel or other suitable material. The stylet may have a plastic or metal hub and may be made of metal, plastic, or other suitable material. The needle shaft may be marked or not with depth marking lines using metric or standard units of measure. Wings may be constructed of plastic or other suitable material. Wings may also be omitted from this design. This needle is suitable for cervical, thoracic, lumbar, and caudal use.

Advantages

From the description above, a number of advantages of the Ice Needle become evident:

a) The larger-diameter proximal shaft allows safe, reliable directional control during injection, enabling the tip to reach its desired target. Smaller-diameter needle shafts can easily divert, penetrating nervous and vascular structures, and organs; causing potential bleeding or other injury.

b) The smaller-diameter distal tip will cause less injury to any nervous and vascular structures, and organs; if these are inadvertently contacted or punctured.

c) Some patients have very narrow epidural spaces. Patients with a history of spine surgery may have significant scarring in this area, obliterating this space. The small tip on this needle will cause only a small defect if it punctures the dural sheath, minimizing spinal fluid leakage. In this way, the likelihood of post dural puncture headache is greatly decreased.

d) The semi-rounded or blunt tip will further minimize tissue trauma. When taken with the previous advantages, the safety of this needle exceeds that of the prior art and of commercially available epidural needles.

e) The design of the Ice Needle is less complex than that of other prior art designs. This will allow ease of manufacture and, in so doing, reduce manufacturing costs.

### Conclusion, Ramifications, and Scope

Accordingly, the reader will see that the Short-Tapered Epidural Injection Needle (Ice Needle) provides a safe, efficient means of placing medications into the epidural space of the cervical, thoracic, lumbar, and sacral spinal areas. It allows reliable guidance to the epidural space while minimizing trauma to any tissues penetrated by the small tip.

While the previous description contains certain specificities, these should not be construed as limitations on the scope of the invention, but rather as an exemplification of one preferred embodiment thereof. Many other variations and combinations are possible. For example, the needle and stylet may be provided in a variety of colors. The needle and stylet need not be made of the same material. The needle may have a variety of proximal openings, such as Luer-Lock. If metal, the components may be of stainless steel, aluminum, or other suitable alloy or metal.

Thus the scope of the invention should be determined by the appended claims and their legal equivalents, rather than by the examples given.

1. An epidural injection needle, comprising:
(a) a proximal hollow hub;
(b) a stylet having an outer diameter approximately equal to the inner diameter of said distal tip;
(c) a proximal elongated tubular shaft extending from the hub, said elongated shaft extending distally;
(d) a distal short-tapered tubular portion, a very short distance from the distal end of the needle;
(e) a distal tip comprising one of a plurality of commercially-available tip types;
(f) whereby said short-tapered portion reduces the gauge size of said needle from said proximal shaft to said distal tip.

2. The needle of claim 1 wherein said distal tubular end has a blunt or semi-rounded tip.

3. The needle of claim 2 wherein said tip is beveled.

4. The needle of claim 1 wherein the hollow hub is of a locking type.

5. The needle of claim 1 wherein the hollow hub is of a non-locking type.

6. The needle of claim 1 wherein means for grip and control of device, such as wings, are included.

7. The needle of claim 1 wherein said proximal tubular shaft is marked with indications of measure.

8. The needle of claim 7 wherein said indications of measure are in metric or standard nomenclature units.

9. A method of injecting medication into the epidural space, comprising:
(a) a larger-gauge proximal elongated tubular shaft extending distally from a proximal hub, enabling good directional stability of said shaft;
(b) a short-tapered tubular portion just proximal to a distal tip, reducing gauge size from said proximal shaft to said distal tip;
(c) said distal tip comprising one of a plurality of commercially-available needle tip varieties for entering said epidural space;
(d) whereby said short-tapered portion reduces the gauge size of the device from said proximal shaft to said distal end, enabling medications to be safely placed into said epidural space.

10. The needle of claim 9 wherein the hub type is locking.

11. The needle of claim 9 wherein said hub type is non-locking.

12. The needle of claim 9 wherein a means for grip and control of device, such as wings, is included.

13. The needle of claim 9 wherein said proximal tubular shaft is marked with indications of measure.

14. The needle of claim 13 wherein said indications of measure are in metric or standard nomenclature units.
